(19) United States
(12) Patent Application Publication
(10) Pub. No.: US 2009/0311303 A1
(43) Pub. Date: Dec. 17, 2009

(54) BIODEGRADABLE IMPLANTS FOR BONE FUSION AND COMPOSITIONS THEREFOR

(75) Inventors: Cameron Lutton, Queensland (AU); Ben Goss, Queensland (AU)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Queensland University of Technology, Brisbane (AU)

(21) Appl. No.: 12/298,481

(22) PCT Filed: Apr. 27, 2007
(86) PCT No.: PCT/AU2007/000551

§ 371 Date: Feb. 26, 2009

(30) Foreign Application Priority Data
Apr. 27, 2006 (AU) 2006902194

(51) Int. Cl. A61L 27/54; A61L 27/58

(52) U.S. Cl. 424/426; 523/114

(57) ABSTRACT

A biodegradable copolymer network, which is particularly suited for use in applications such as interbody implants for bone fusion, is provided. The biodegradable copolymer network of the present invention is biodegradable yet provides sufficient mechanical strength for use in procedures such as spinal fusion. Also provided are methods of producing said biodegradable copolymer network, methods of delivery of biologically active substances, implants comprising said biodegradable copolymer network and methods of administering bioactive and in particular, osteoinductive, substances.

### FIELD OF THE INVENTION

The present invention relates generally to methods for preparation of biodegradable, crosslinlced co-polymer networks of functionalized anhydrides and a non-biodegradable component for use in orthopedic and dental applications.

The present invention relates to biodegradable implants for bone fusion and compositions therefor. In particular, but not exclusively, the present invention relates to biodegradable implants for use as cages, rods, plates, screws, nails and bone graft substitutes. The present invention also relates to a method of delivery of biologically active substances through a series of separate sites in the implant with different degradation rates. The cumulative release profile form these sites results in an appropriate release for the active molecules.

### BACKGROUND TO THE INVENTION

Bone fusion techniques are conventionally used on and around the spine and fractures and bone graft is often required during hip and spine surgery, as well as during fracture fixation. These techniques currently all involve the use of metallic or non-degradable polymer cages, rods screws and nails.

For example, of the 65 million people in the United States experiencing lower back pain, approximately 151,000 undergo fusion of the lumbar spine each year. Surgery such as spinal fusion (arthrodesis) is used to minimise or eliminate the pain caused and typically involves immobilising two or more vertebrae. Current techniques may utilise poly ether ether ketone (PEEK)/carbon fibre or metallic interbody cages, typically in the form of cylinders or wedges, that are placed in the disc space between the vertebrae. The cages comprise cavities that allow a bone graft to be placed in the cage to encourage bone to grow through and around the cage resulting in fusion of the vertebral bodies.

A further example is for the subset of trauma patients who undergo intra medullary nailing. In this procedure a titanium nail is inserted into the medullary cavity of a long bone in order to provide a stable bridge over the fracture ends. Regularly the nail is removed after the fracture has healed, requiring a second operation.

There are many other examples of orthopaedic procedures that utilise cages plates screws and nails made from metal or non-degradable polymers.

However, such implants present a range of problems. Such metals or non-degradable polymers are permanent and remain in the body, even when the bone growth is sufficient to render the implant superfluous. Retention of the implants in the body can also lead to biocompatibility problems. A specific problem is that the footprint of the cages reduces the vertebral end plate area available for bone fusion. A further problem that is sometimes encountered is that since the implants provide sufficient immobilisation of the vertebrae or fracture surface bone growth is redundant/inhibited and solid fusion is not achieved (Pseudarthrosis). The long-term effects of metallic cages on the spine are not yet known, but reported complications to date include migration, adjacent level degeneration, subsidence from stress shielding, stenotic myelopathy and artefacts in postoperative radiological assessment. Thus there is a need to develop implants that biodegrade is a controlled manner to alleviate the long term problems of permanent implants.

Bone graft is often used in conjunction with these above mentioned orthopaedic procedures. Conventional methods for the treatment of large bone defects consist of harvesting autogenous bone, usually from the pelvis, because this site yields the best quality and largest quantity of bone. Despite its applicability for grafting, pelvic harvesting is associated with significant morbidity, post operative pain and complications. Arrington et al found a 15% (10% minor and 5% major) complication rate, with major complications including infections, abdominal herniation, neurological injuries and iliac wing fractures^{[4]}. An alternative to autogenous bone is cadaveric allograft bone. Although there is a ready supply of this material there are a number of drawbacks associated with its use. In particular there are decreases in mechanical properties, increased risks of disease transmission and higher rates of non-union, when compared with autograft ^{[5]}. Sterilization of allograft usually destroys all pre-existing biological function leaving it as an osteoconductive material only. The complications associated with autograft and the drawbacks of allograft highlight the need for synthetic biodegradable materials that can act as graft extenders or substitutes to reduce graft site morbitity and pain, or eliminate the operation all together.

A range of biodegradable polymers have been developed including degradable polyesters such poly (L-lactic acid), poly(glycolic acid), and poly(lactic-co-glycolic acid) as well as, polyfumarates polyhydroxyalkanoates and polyorthoesters for use as structural orthopaedic implants as well as bone graft extenders. These polymers, however, lack many properties necessary for restoring fanction in high load-bearing bone applications, as they undergo homogeneous, bulk degradation which is detrimental to the long-term mechanical properties of the material and leads to a large burst of acid products near the end of degradation. In contrast, surface eroding polymers (such as polyanhydrides) maintain their mechanical integrity by preserving the molecular weight of the polymer and exhibit a gradual loss in size which permits bone ingrowth. However, current linear and crosslinlked polyanhydride systems have mechanical properties that are inappropriate for load bearing applications. (U.S. Pat. No. 5,902,599).

Another major drawback for these systems is a lack of biological activity. Attempts to address the aforementioned problem of inhibited or insufficient bone growth have included the utilization of osteoinductive substances, such as growth factors. Examples of growth factors include bone morphogenetic proteins-2 and 7 (BMP-2 and BMP-7), insulin-like growth factor-1 (IGF-1), Platelet Derived Growth Factor (PDGF), Vascular Endothelial Growth Factor (VEGF), Fibroblast Growth Factor (FGF) and Transforming Growth Factor-β1 (TGF-β1). For example, BMPs have been demonstrated to accelerate spinal fusion, IGF-1 stimulates the replication of osteoblasts and the synthesis of bone matrix and TGF-β1 regulates different cell types involved in bone remodelling and fracture healing. Kandziora, T. et al. (Bone morphogenetic protein-2 application by a PDLLA-coated interbody cage: in vivo results of a new carrier for growth factors, J Neurosurg (Spine 1) Vol.97 :40-48, July 2002) and Schmidmaier, G. et al. (Local application of growth factors (IGF-1 and TGF-β1) from a biodegradable PDLLA coating of osteosynthetic implants accelerates fracture healing in rats, Bone Vol.28, No.4:341-350, April 2001) disclose the use of a biodegradable coating of PDLLA on interbody fusion cages as an effective delivery system for growth factors. Related United States Patent Application No. US 2006/0039947 in the name of Schmidmaier, G. et al. discloses additional growth factors that can be employed.

Whilst the combination of biodegradable implants and growth factors represent an advancement over metallic and carbon fibre cages and over biodegradable implants alone, there is a need to control the release of such growth factors. For example, a disadvantage of many growth factors (including BMP-2) is rapid inactivation in vivo after 20-30 minutes. Furthermore, the effect of growth factors can be dose-dependent and can have opposite effects at high or low doses. Therefore, localised, controlled and continuous release of the growth factors is necessary to accelerate implant incorporation and stimulate fracture healing.

Attempts to control the release of drugs from micro-reservoir devices are disclosed by Richards Grayson, A.C. et al. (Molecular release from a polymeric micro-reservoir device: influence of chemistry, polymer swelling and loading on device performance, J Biomed Mater Res 69A: 502-512, 2004, and Multi-pulse drug delivery from a resorbable polymeric microchip device, Nature Materials, Vol 2, Nov. 2003). The devices comprise a plurality of micrometre-scale reservoirs that can be filled with a chemical for release into the body. The reservoirs are sealed with a thin, biodegradable membrane of PLGA and the contents of the reservoirs are released upon degradation of the membrane. Whilst a time of release of the payload from each reservoir can be controlled by varying the molecular masses or the materials of the membranes, all of the payload is released from the reservoirs at once resulting in a pulsed release of the active ingredient rather than the desired continuous release over time.

In this specification, the terms “biodegradable”, “degradable” “resorbable” and “bioresorbable” are defined as the biologic elimination of the products of degradation by metabolism and/or excretion and the term “bioerodible” is defined as the susceptibility of a construct to mass loss by biodegradation over time.

In this specification, the terms “comprises”, “comprising” or similar terms are intended to mean a non-exclusive inclusion, such that a method, system or apparatus that comprises a list of elements does not include those elements solely, but may well include other elements not listed.

### OBJECT OF THE INVENTION

It is an object of the present invention to provide an improved orthopaedic implant composition that addresses or at least ameliorates one or more of the aforementioned prior art problems or provides a useful alternative.

### SUMMARY OF INVENTION

In one form, although it need not be the only or indeed the broadest form, the invention resides in a biodegradable copolymer network for an implant, said biodegradable copolymer network comprising monomers or pre-polymers having functional groups polymerisable by a free radical mechanism, at least one of the pre-polymers comprising a hydrolytically cleavable functional group, wherein said pre-polymers are co-polymerized with an enhancing monomer or pre-polymer that enhances the mechanical properties of the biodegradable copolymer network.

By “pre-polymers” it is meant either short-chain polymers or monomers.

Suitably, the at least one of the pre-polymers comprises a degradable component in the form of a hydrolytically cleavable functional group.

Suitably, the enhancing monomer or pre-polymer that enhances the mechanical properties of the biodegradable copolymer network comprises an enhancing component.

Suitably, the polymerisable aspect of the degradable component is selected from the following groups: acrylates, methacrylates, alkenes and the hydrolytically cleavable functional group is selected from the following groups: anhydrides, esters, carbonates, amino carbonates, ortho-esters.

Preferably, the hydrolytically cleavable functional group is an anhydride.

In another form, the invention resides in a method of producing a biodegradable copolymer network for an implant including the steps of:
synthesizing pre-polymers by nuecleophilic substitution, said pre-polymers having functional groups polymerisable by a free radical mechanism and at least one pre-polymer having a hydrolytically cleavable functional group; and
co-polymerising the pre-polymers with an enhancing monomer or polymer that enhances the mechanical properties of the biodegradable copolymer network.

Suitably, the enhancing component includes an acrylate, methacrylate or otherwise unsaturated functional group.

Preferably, the enhancing component is a methacrylate and preferably methyl methacrylate.

Suitably, the a combination of the degradable component and the enhancing component are copolymerized at elevated temperatures, using free radical techniques, obvious to anyone skilled in the art.

Preferably the combination is thermally polymerized using benzoyl peroxide catalyst at 70° C. and post cured at 120° C.

Suitably, the degrading component includes di- and/or tri-methacrylated acid anhydrides.

Suitably, the enhancing component includes di- and/or tri-methacrylated compounds.

Suitably, the co-polymer further comprises inorganic reinforcement, such as calcium hydroxyapatite and/or barium hydroxyapatite.

Suitably, the biodegradable copolymer network further comprises one or more osteoinductive substances, such as biological growth factors, such as, but not limited to, BMP-2, BMP-7, IGF-1, TGF-β1.

In a further form, the invention resides in an implant at least partially made from the above biodegradable copolymer network.

Suitably, the implant comprises any amount of the above biodegradable copolymer networks.

Suitably, the implant is selected from the group consisting of a spinal fusion interbody cage, a intramedullary nail, a screw, a plate and a bone graft substitute.

Suitably, the implant comprises at least one, and preferably a plurality of, cavities, recesses or voids for receiving two or more biodegradable copolymer networks comprising one or more bioactive substances, such as biological growth factors, osteoinductive substances or antibiotics.

Preferably, multiple biodegradable copolymer networks having differing degradation rates are provided in the cavities, recesses or voids resulting in a cumulative release profile for the controlled, continuous and localized delivery of the bioactive substances.

The cavities, recesses or voids may contain one or more biodegradable copolymer networks such as, but not limited to, PLGA, PCL, polyanhydride as well as biodegradable copolymer compositions disclosed in this application.

The cavities, recesses or voids may comprise one or more bioactive substances, such as, but not limited to, BMP-2, IGF-1, TGF-β1, epidermal growth factor (EGF), fibroblast growth factor (FGF), platelet-derived growth factor (PDGF), gentamicin or tobramicin.

Suitably, the cavities, recesses or voids are provided on at least one surface of the implant and/or within the body of the implant.

In a yet further form, the invention resides in an implant comprising at least one, and preferably a plurality of, cavities, recesses or voids for receiving two or more biodegradable copolymer networks comprising one or more bioactive substances, such as biological growth factors.

Preferably, the biodegradable copolymer networks comprising one or more bioactive substances are polymerized in the cavities, recesses or voids of the implant.

Even more preferably, the biodegradable copolymer networks comprising one or more bioactive substances are polymerized in the cavities, recesses or voids of the implant by photopolymerisation.

Suitably, the implant may be formed from PEEK/carbon fibre, titanium or other materials.

In one particular form the invention resides in a method of performing spinal fusion, said method including the step of administering an implant formed from a biodegradable copolymer network of the present invention.

In yet another form, the invention resides in a method of preparing implants for use in spinal fusion, fracture fixation, and bone void filling implant comprising one or more cavities for receiving one or more biodegradable copolymer networks comprising one or more bioactive substances to an animal to thereby perform bone healing and growth.

In yet a further form, the invention resides in a method of administering a bioactive substance in a controlled, continuous and localized manner, said method including the step of administering an implant to an animal, said implant comprising a biodegradable copolymer network comprising one or more pre-polymers having a functional group polymerisable by a free radical mechanism, wherein at least one of the pre-polymers comprising a hydrolytically cleavable functional group; and wherein the implant comprises one or more cavities at least partially filled with a biodegradable copolymer network that contains one or more bioactive substances.

In yet a further form, the invention resides in a method of administering an bioactive substance in a controlled, continuous and localized manner, said method including the step of administering an implant to an animal, said implant comprising one or more cavities at least partially filled with a biodegradable copolymer network comprising one or more bioactive substances, said biodegradable copolymer network comprising one or more pre-polymers having a functional group polymerisable by a free radical mechanism, wherein at least one of the pre-polymers comprising a hydrolytically cleavable functional group, said pre-polymers co-polymerized with an enhancing polymer that enhances the mechanical properties of the biodegradable copolymer network; and
wherein said implant is formed from a predominantly non-biodegradable copolymer network.

In any of the aforementioned forms, the animal is selected from the group consisting of humans, domestic livestock, laboratory animals, companion animals and performance animals.

Preferably, the animal is a human.

Further features and forms of the present invention will become apparent from the following detailed description.

### BRIEF DESCRIPTION OF THE DRAWINGS

By way of example only, preferred embodiments of the invention will be described more fully hereinafter with reference to the accompanying drawings, wherein:

FIG. 1 is a flowchart illustrating a method of producing a biodegradable copolymer network for an implant according to an embodiment of the present invention;

FIG. 2 is a front perspective view of a biodegradable implant according to an embodiment of the present invention;

FIG. 3 is an enlarged perspective view of an edge surface of a biodegradable implant according to another embodiment of the present invention;

FIG. 4 is a front perspective view of a biodegradable implant according to a further embodiment of the present invention;

FIG. 5 is an enlarged perspective view of an edge surface of the biodegradable implant of FIG. 4 with the elongate recesses unfilled;

FIG. 6 shows the biodegradable implant of FIG. 5 with the elongate recesses filled with different biodegradable copolymer networks;

FIG. 7 (A) and (B) is graphical representation of results of mechanical testing for various ratios of MSA:MMA;

FIG. 8 represents a calculation of the maximum load sustainable by a spinal cage of different sizes prepared from these materials compared to the load measured in a human intervertebral disc segment;

FIG. 9 shows a gross section of sheep femur into which has been implanted a biodegradable cage;

FIG. 10 is a histological section of the bone adjacent to the implant site of FIG. 9;

FIG. 11 is a flowchart of illustrating a method of polymerisation of a drug delivery system on an implant according to an embodiment of the present invention; and

FIG. 12 (A) and (B) show that the degradation rate of these materials can be controlled by altering the composition; and

FIG. 13 (A) and (B): FIG. 13(A) shows the individual release profiles for the dye and FIG. 13(B) shows an optimised release profile from a combination of the delivery systems.

### DETAILED DESCRIPTION OF THE INVENTION

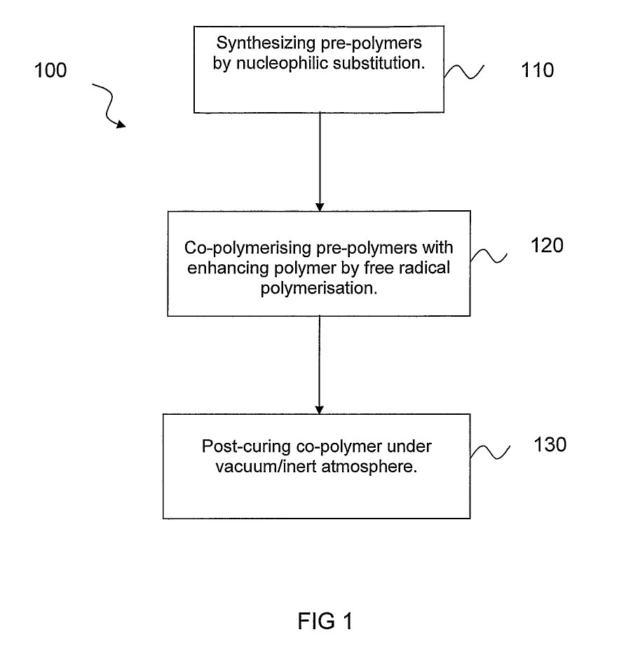

Referring to the flowchart in FIG. 1, and in accordance with an aspect of the present invention, there is provided a method 100 of producing a biodegradable copolymer network for an orthopedic implant. The method includes step 110 of synthesizing pre-polymers by condensation and preferably by nucleophilic substitution. The pre-polymers have functional groups polymerisable by a free radical mechanism and the pre-polymers can be selected from the following group: acrylates, methacrylates, alkenes. At least one of the pre-polymers comprises a hydrolytically cleavable functional group selected from the following group: anhydrides, esters, carbonates, amino carbonates, ortho-esters.

The method 100 includes step 120 of co-polymerising the pre-polymers with an enhancing polymer that enhances the mechanical properties of the biodegradable copolymer network. In the context of the present invention, “enhances”, “enhance”, “enhancement” or “enhanced” refers to an increase, improvement or otherwise augmentation of the mechanical properties of the biodegradable copolymer network. By way of example only, enhancement of mechanical properties may manifest as an increase in compressive strength, but is not limited thereto.

According to one embodiment, the enhancing polymer is a methacrylate and in a preferred embodiment, the enhancing polymer is methyl methacrylate (MMA). According to a preferred embodiment, the co-polymerisation process uses benzoyl peroxide as an initiator and takes place at a substantially constant temperature of between 50° C. and 90° C. for between approximately 0.5 and 3.0 hours to form biodegradable networks that degrade by surface erosion. In an alternative embodiment, co-polymerisation of the pre-polymers occurs at a temperature that increases over time from 65° C. to 120° C. over a period of up to 16 hours. In this embodiment, the benzoyl peroxide is 0.25 wt %, but between 0.1 wt % and 5.0 wt % benzoyl peroxide could be utilized.

Step 130 of the method 100 includes post curing the co-polymer under vacuum or in an inert atmosphere at a substantially constant temperature of between 110 and 140° C. for between approximately 0.1-2 hours. In a preferred embodiment, post curing is performed at 120° C. for 0.5 hours.

The degradable component of the pre-polymers can include di- and/or tri-methacrylated acid anhydrides. Suitable forms of the di-methacrylated acid anhydride are aliphatic di-acid anhydrides including, but not limited to 1,6-hexandioic acid anhydride 1,7-heptanedioic acid anhydride, 1-8 octanedioic acid anhydride, 1,9-nonanedioic acid anhydride, or 1,10-decanedioc acid anhydride, phenyl di-acid anhydrides including, but not limited to 2,2′-[1,4-phenylenebis(oxy)]bis-Acetic acid anhydride, 2,2′-[1,4-phenylenebis(oxy)]bis-propanoic acid anhydride, 2,2′-[1,4-phenylenebis(oxy)]bis-butanoic acid anhydride, or suitable substitutions thereof.

Suitable forms of the tri methacrylated acid anhydride are aliphatic tri acid anhydrides including, but not limited to 2-hydroxy-1,2,3-propane tricarboxylic acid anhydride, 1,2,3-propane tricarboxylic acid anhydride, 4-carboxymethyl 1,6 hexanedioic acid anhydride, 4-carboxymethyl 1,7-heptanedioic acid anhydride, 4-carboxymethyl 1-8 octanedioic acid anhydride, 4-carboxymethyl 1,9-nonanedioic acid anhydride, 4-carboxymethyl 1,10-decanedioic acid anhydride or suitable substitutions thereof.

The enhancing component of the pre-polymers can include uni, di and tri functional acrylates, methacrylates, vinyl unsaturated compounds or other compounds polymerisable through free radical mechanisms. Examples of these materials include, but are not limited to, methyl methacrylate, methyl acrylate, styrene, butyl methacrylate, 1,1,1 -trimethylol propane trimethacrylate.

In one embodiment, the biodegradable co-polymer further comprises inorganic reinforcement, such as, but not limited to, calcium hydroxyapatite and/or barium hydroxyapatite.

According to one embodiment, the biodegradable copolymer network further comprises one or more osteoinductive substances, such as biological growth factors. Examples of suitable forms of such growth factors include, but are not limited to, bone morphogenetic protein-2 and 7 (BMP-2, BMP-7), insulin-like growth factor-1 (IGF-1) and transforming growth factor-β1 (TGF-β1). The growth factors are mixed in with the degradable composition prior to polymerization in the cavity.

According to another embodiment, the biodegradable copolymer network further comprises a radio-opacifier such as, but not limited to, hydroxyapatite or barium hydroxyapatite.

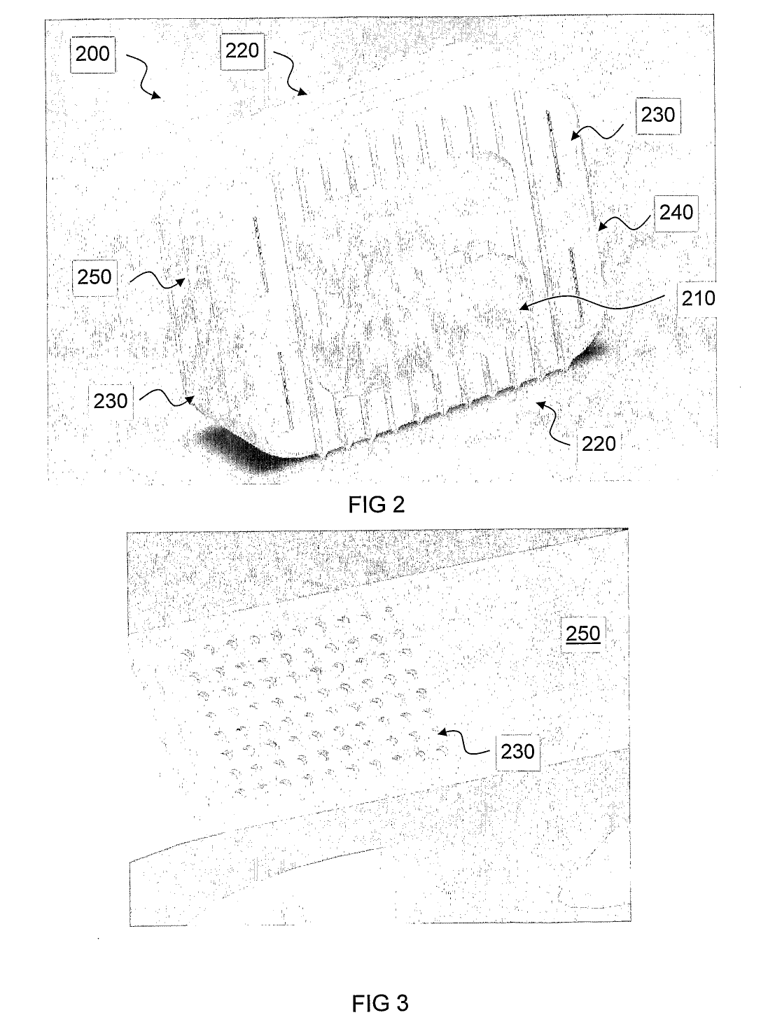

With reference to FIGS. 2-6, according to another aspect of the present invention, there is provided an implant 200 at least partially made from the aforementioned biodegradable copolymer network. Depending on the particular application of the implant and the specific characteristics required, such as the degradation rate, the implant 200 can comprise an amount of the biodegradable copolymer network. By way of example, the implant 200 may comprise 25-100% of the biodegradable copolymer network. Where the implant does not comprise 100% of the biodegradable copolymer network of the present invention, the remainder of the implant can be constituted from one or more of the following materials: calcium hydroxyapatite, barium hydroxyapatite, titanium, PEEK/carbon fibre composite.

Although the implant 200 has applications as a biodegradable spinal implant in the form of a spinal fusion interbody cage for use in cervical, thoracic and lumbar fusion surgery, the inventors envisage that an implant of the present invention could be utilized in other surgical techniques that require a biodegradable implant exhibiting the mechanical strength characteristics described below (See ‘Examples’ section). The size and shape of the implant 200 will depend on the particular application. For a spinal cage application the implant typically comprises one or more cavities 210 to allow bone graft to grow through the implant and a plurality of surface serrations 220 to grip the bone to which the implant is affixed and to help prevent slipping of the implant 200, as is known from the prior art.

According to the embodiment shown in FIG. 2, the implant 200 comprises at least one, and preferably a plurality of, cavities, recesses or voids 230 for receiving the aforementioned biodegradable copolymer networks comprising one or more bioactive substances, such as biological growth factors and osteinductive substances. The cavities, recesses or voids 230 are provided on at least one face surface 240 and/or at least one edge surface 250 of the implant and/or within the body of the implant. The particular selection will depend on, for example, the locality in which bone growth is to be promoted and the desired rate of release of the osteoinductive substances. FIG. 2 shows a plurality of elongate recesses 230 in at least one face surface 240 and in at least two edge surfaces 250 of the implant 200. In this embodiment, the elongate recesses 230 are substantially parallel with a longitudinal axis of the edge surface 250 and the elongate recesses 230 in the at least one face surface 230 are substantially vertical and substantially parallel with the serrations 220.

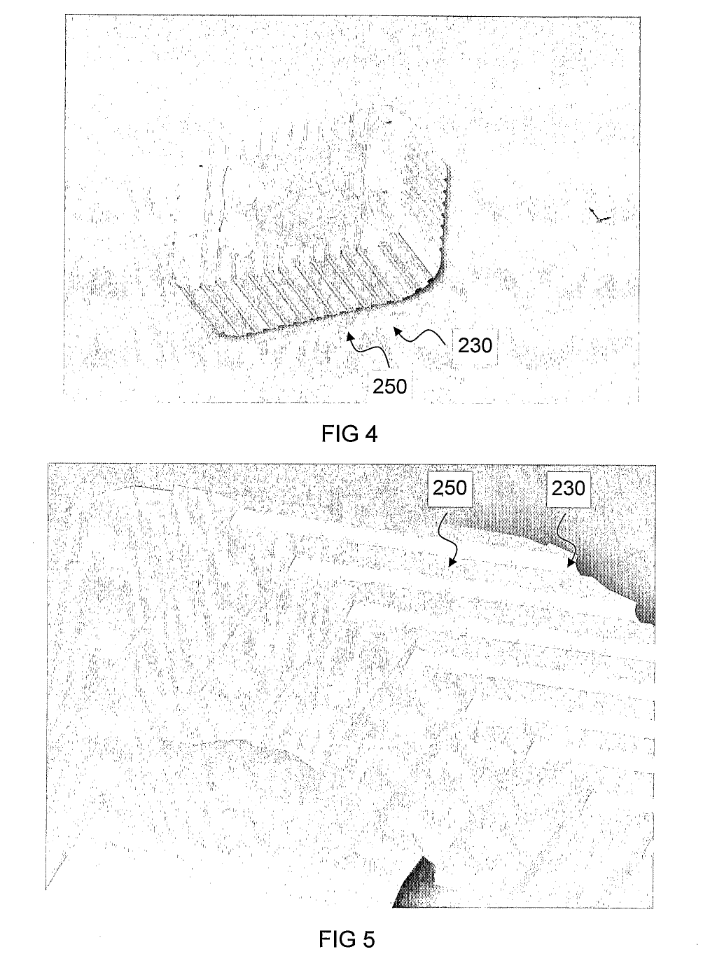

FIG. 3 shows a regular array of substantially circular recesses or wells 230 in an edge surface 250 of the implant 200. In contrast to FIG. 2, FIG. 4 shows an implant 200 in which the elongate recesses 230 in the edge surfaces 250 are substantially perpendicular to the longitudinal axis of the edge surfaces 250.

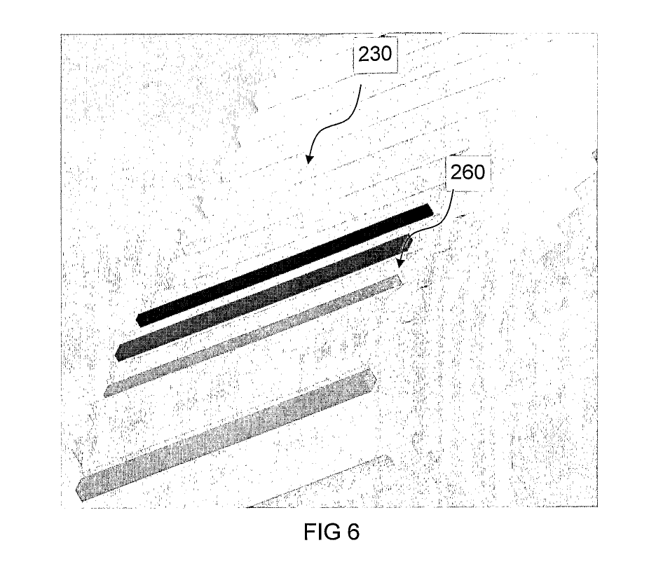

FIG. 5 shows an enlarged view of the empty elongate recesses 230 in one of the edge surfaces 250 of the implant 200 and FIG. 6 shows the same edge surface 250 in which the elongate recesses 230 are filled with multiple biodegradable copolymer networks 260 comprising one or more bioactive substances, such as biological growth factors. The differing degradation rates of the multiple compositions results in a cumulative release profile for the controlled, continuous and localized delivery of the bioactive substances. According to one embodiment, the one or more biodegradable copolymer networks comprising one or more bioactive substances are polymerized in situ in the cavities, recesses or voids of the implant.

A person of skill in the art will readily appreciate that in embodiments which include bioactive substances polymerized in situ, preparation of the co-polymer by free radical polymerisation should be performed by a process that preserves the integrity of the bioactive substance. By way of example only, photopolymerisation using a photoinitiator and UV light with a wavelength between 300 nm-400 nm, to generate a free radical is readily amenable to production of a biodegradable copolymer network of the present invention which comprises an bioactive substance in the form of a protein, but is not limited thereto.

Referring to the flowchart in FIG. 11, and in accordance with an aspect of the present invention, there is a method 300 of photopolymerisation of a drug delivery system on an implant. The method includes the step of preparing a drug delivery formulation. As hereinbefore described, it is envisaged that the drug delivery formulation will comprise a mixture of pre-polymers for formation of a biodegradable copolymer network, a photoinitiator and a drug. Typcially, although not exclusively, the drug is an osteoinductive substance, angiogenic substance or antibiotic substance. According to one preferred embodiment, the photoinitiator is 2,2-dimethoxy-2-phenylacetophenone (DMPA) at about 0.5 wt % but between 0.1 and 5% DMPA could be utilised.

The method 300 further includes step 320 of applying the drug delivery formulation to an implant. Generally, application to an implant will be by means of mechanical dispersion such as pipetting although is not limited thereto.

Step 330 of the method 300 includes generating a free radical by photopolymerising the pre-polymers under exposure to UV light at a wavelength of between 300-400 nm. Photo-polymerisation thereby results in production of an implant having a biodegradable copolymer network comprising bioactive substances which are released in a controlled, sustained and localised manner.

In various embodiments of the implant 200, the one or more biodegradable copolymer networks comprising one or more bioactive substances that fill or at least partially fill the cavities, recesses or voids 230. The various biodegradable copolymer networks protect the bioactive substances and as the compositions degrade in the body, a controlled, continuous and localized release of the bioactive substance(s) is provided, thus avoiding the pulsed delivery of some of the prior art devices. In other embodiments, the cavities, recesses or voids can contain different biodegradable copolymer networks such as, but not limited to, PLGA, poly (epsilon-caprolactone)(PCL), and the free radical polymerisable compositions described above.

In another embodiment, the cavities, recesses or voids 230 of the implant 200 comprise one or more bioactive substances examples of which are listed above.

Generally, the level of anhydride in the composition can be increased to increase the rate of degradation of the composition. For example, the rate of degradation of the one or more compositions comprising the bioactive substances can be selected to be faster than the degradation rate of the implant 200 such that the bioactive substances are released whilst the load bearing properties of the implant are maintained.

It should be appreciated that the aforementioned protection, and controlled, continuous and localized method of delivery, of the bioactive substance(s) can be utilized in conventional implants and not merely in the implants of the present invention made from the aforementioned biodegradable copolymer networks. Hence, it will be readily appreciated that one broad application of the present invention is a drug delivery system.

### EXAMPLES

### Example 1

### Preparation and Polymerisation of Methacrylated Sebacic Acid Anhydride-co-Methyl Methacrylate Networks

Methacrylated sebacic acid anhydride (MSA) was prepared by the addition of 24 g (2.1 mole equivalents) of methacrylic anhydride to 15 g of sebacic acid. The resulting slurry was stirred at 75° C. under argon for 1.5 hours with the constant removal of methacrylic acid. After cooling the monomer was separated from any residual methacrylic anhydride via solvent extraction using hexane. A 70MSA:30MMA mole ratio network was prepared by mixing 4.2 g of MSA and 0.364 g of methyl methacrylate. The solution was warmed at 50° C. for 5 minutes and 0.5% wt benzoyl peroxide was added. The mixture was transferred to polymerisation tubes and cured under argon at 70° C. for 30 minutes before undergoing a final cure at 140° C. for 10 minutes. The polymers were then allowed to relax for several days at room temperature before testing. Polymers with varying ratios of pre-polymers were synthesised using this method and testing accordingly.

### Example 2

### Preparation and Polymerisation of Methacrylated Adipic Acid Anhydride-co-Methyl Methacrylate-co-Trimethylpropane Trimethacrylate Networks

Methacrylated adipic acid anhydride (MAA) was prepared by the addition of 34 g (2.1 mole equivalents) of methacrylic anhydride to 15 g of adipic acid. The resulting slurry was stirred at 75° C. under argon for 1.5 hours with the constant removal of methacrylic acid. After cooling the monomer was separated from any residual methacrylic anhydride via solvent extraction using hexane. A 90MAA:7.5MMA:2.5TMPTM mole ratio network was prepared by mixing 5.4 g of MAA, 0.165 g of methyl methacrylate and 0.16 g of trimethylpropane trimethacrylate. The solution was warmed at 50° C. for 5 minutes and 0.5% wt benzoyl peroxide was added. The mixture was then transferred to polymerisation tubes and cured under argon at 70° C. for 30 minutes before undergoing a final cure at 140° C. for 10 minutes. The polymers were then allowed to relax for several days at room temperature before testing.

### Example 3

### Preparation and Polymerisation of Methlacrylated Sebacic Acid Anhiydride-co-Methacrylated Tricarballic Acid Anhydride Networks

Methacrylated sebacic acid anhydride (MSA) was prepared by the addition of 24 g (2.1 mole equivalents) of methacrylic anhydride to 15 g of sebacic acid. The resulting slurry was stirred at 75° C. under argon for 1.5 hours with the constant removal of methacrylic acid. After cooling the monomer was separated from any residual methacrylic anhydride via solvent extraction using hexane. Methacrylated tricarballic acid anhydride was prepared and purified in a similar manner. A 97.5 MSA:2.5 MTCA mole ratio network was prepared by mixing 5.8 g of MSA and 0.17 g of MTCA. The solution was warmed at 50° C. for 5 minutes and 0.5% wt benzoyl peroxide was added. The mixture was transferred to polymerisation tubes and cured under argon at 70° C. for 30 minutes before undergoing a final cure at 140° C. for 10 minutes. The polymers were then allowed to relax for several days at room temperature before testing.

### Example 4

### Preparation and Polymerisation of Methlacrylated Adipic Acid Anhydride-co-Methacrylated Sebacic Acid Anhydride-co-Styrene Networks

Methacrylated adipic acid anhydride (MAA) and methacrylated sebacic acid anhydride were prepared by the addition 2.1 mole equivalents of methacrylic anhydride to 15 g of the corresponding diacids. The resulting slurries were stirred at 75° C. under argon for 1.5 hours with the constant removal of methacrylic acid. After cooling the monomers were separated from any residual methacrylic anhydride via solvent extraction using hexane. A 70MSA:20MAA:10Styrene mole ratio network was prepared by mixing 4.2 g of MSA, 1 g of MAA and 0.19 g of Styrene. The solution was warmed at 50° C. for 5 minutes and 0.5% wt benzoyl peroxide was added. The mixture was then transferred to polymerisation tubes and cured under argon at 70° C. for 30 minutes before undergoing a final cure at 140° C. for 10 minutes. The polymers were then allowed to relax for several days at room temperature before testing.

### Mechanical Testing

All polymers were cut into cylinders 5 mm in diameter and 10 mm high and compression tested at a rate of 2 mm/min.

### Degradation Studies

All polymers were cut into cylinders 5 mm in diameter and 10 mm in height and placed in phosphate buffered saline at 37° C. Samples were weighed to dryness in a vacuum oven.

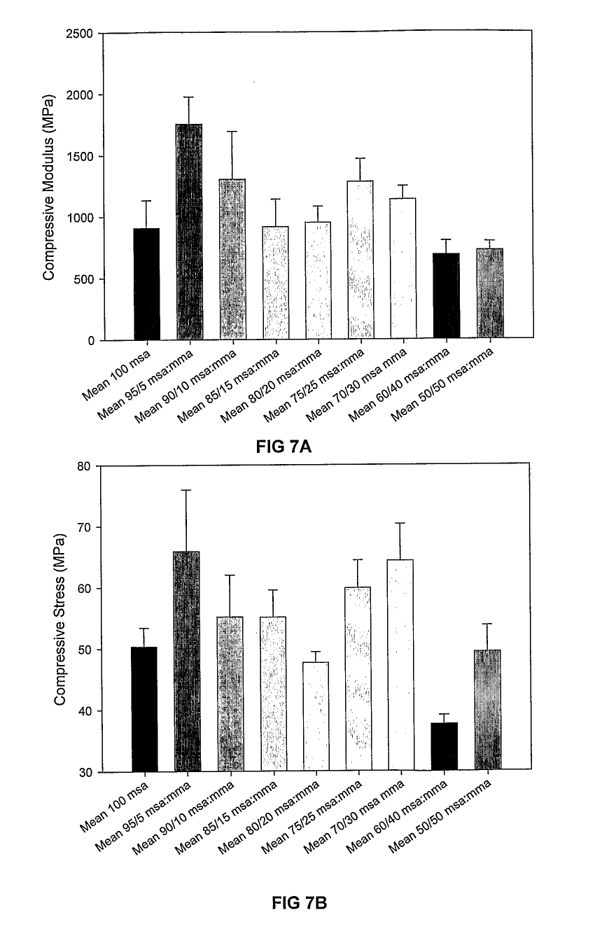

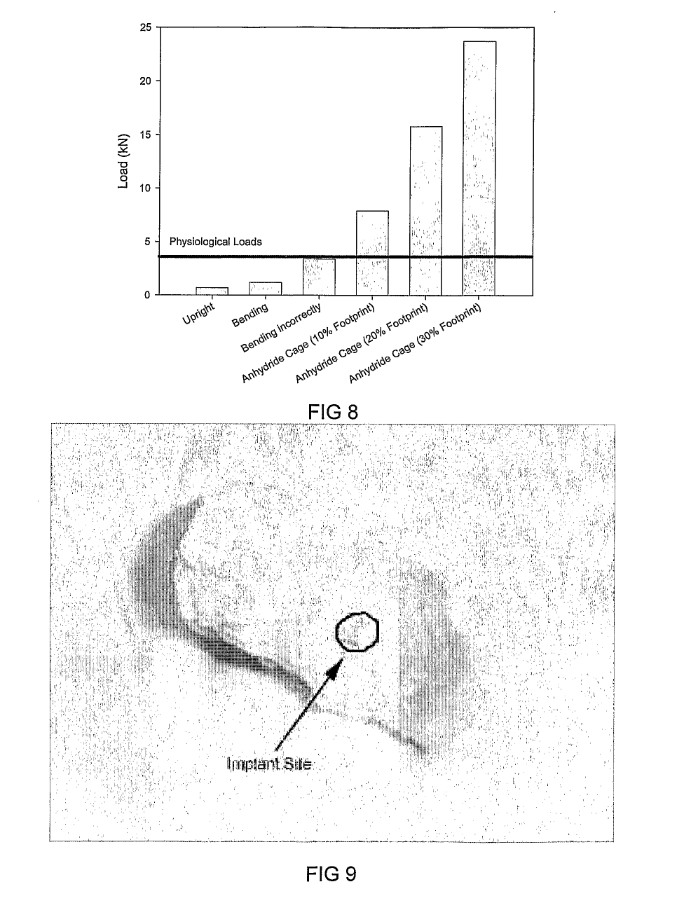

The representative results of mechanical testing are presented in FIG. 7A and 7B. This shows that addition of the enhancing component, in this example methyl methacrylate in varying amounts significantly improves the mechanical performance of the material. The compressive modulus ranges from 0.9 Gpa to 1.7 GPa and the strength from 47 MPa to 64 MPa. These properties represent sufficient mechanical strength for orthopaedic applications. FIG. 8 represents a calculation of the maximum load sustainable by a spinal cage of different sizes prepared from these materials compared to the load measured in a human intervertebral disc segment. This also indicates the suitability of these materials in the aforementioned applications.

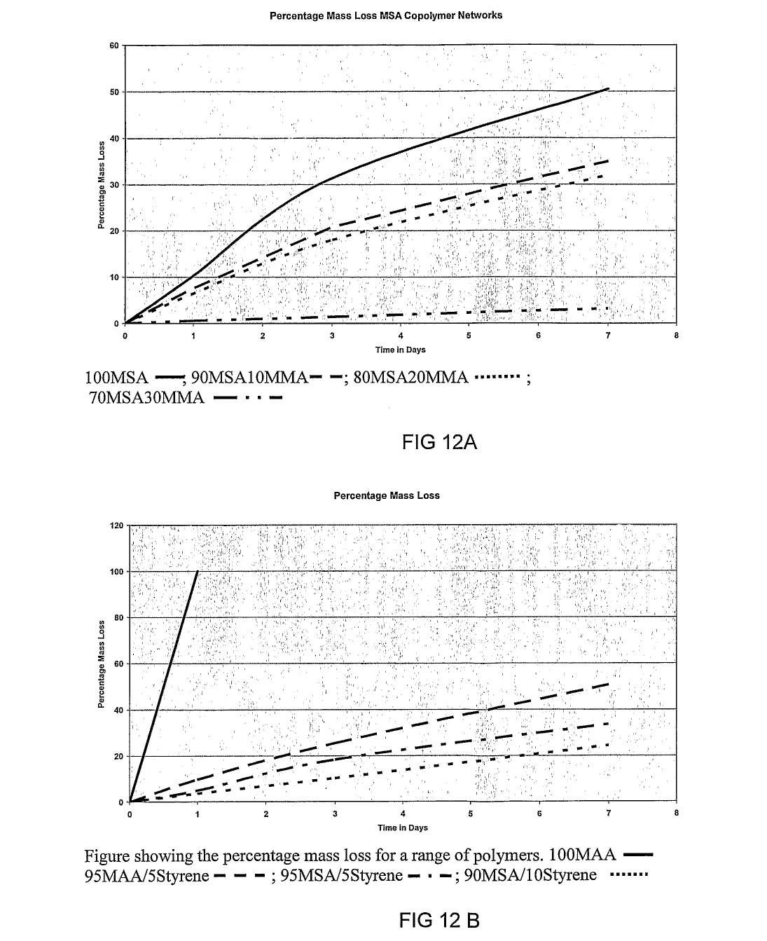

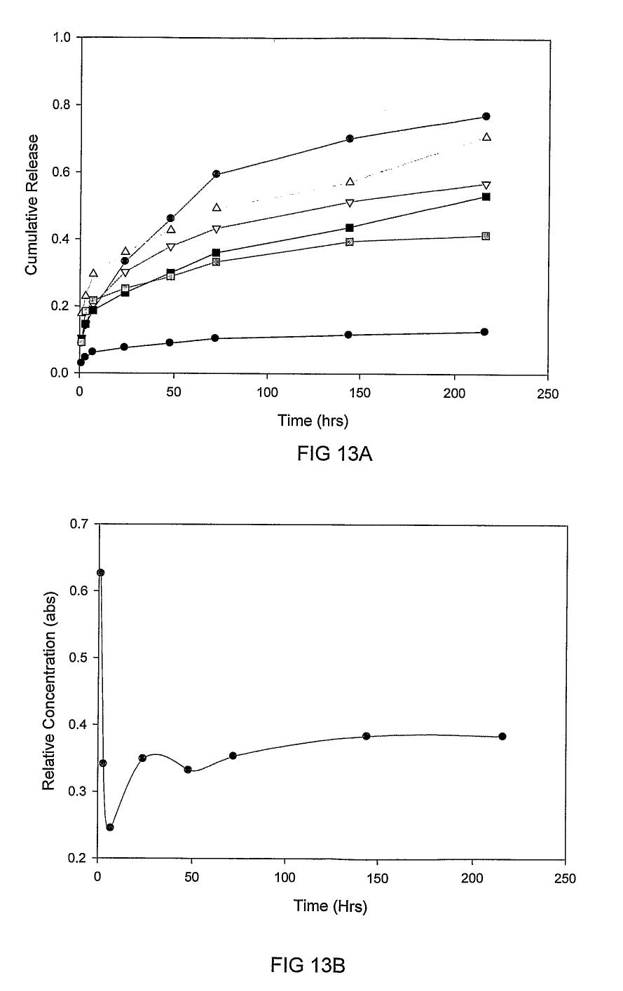

FIGS. 12 (A) and 12 (B) show that the degradation rate of these materials can be controlled by altering the composition. This also shows that the incorporation of non degradable enhancers does not prevent the material from undergoing surface degradation.

### Example 5

### Photopolymerisation of a Delivery System on a Spinal Cage

Six solutions of methacrylic anhydride and methyl methacrylate were prepared in various molar ratios such that the concentrations of methacrylic anhydride ranged from 100% to 50%. The resulting monomer mixtures had a weight of 1 g. To each solution 0.5% wt of 2,2-dimethoxy-2-phenylacetophenone (DMPA), a photoinitiator, was added. 200 uL of each solution was added by pipette onto grooves on a spinal cage and photopolymerised using an Ultra Violet lamp with a wavelength of 300 nm for 5 minutes. This demonstrates the feasibility of photo-polymerisation of a range of biodegradable materials on an implant surface.

### Example 6

### Delivery Study

In order to assess the potential efficacy of the biodegradable copolymer networks as a drug delivery system, elution profile of a dye from various compositions was determined. Six solutions of methacrylic anhydride and methyl methacrylate were prepared in various molar ratios such that the concentrations of methacrylic anhydride ranged from 100% to 50%. The resulting monomer mixtures had a weight of 1 g. To each solution 0.5% wt of 2,2-dimethoxy-2-phenylacetophenone (DMPA), a photoinitiator, was added. To each solution 0.01 g of fluorescein (3×10⁻⁵ moles), a fluorescent dye, was dissolved. The resulting monomer/dye combinations were cast into disks lOmm in diameter and 3mm high and photopolymerised using Ultra Violet lamp with a wavelength of 300 nm for 5 minutes. All disks were placed in 25ml of PBS at 37° C. The resulting release profile of the dye was characterised using UV-Vis spectroscopy at 488 nm. FIG. 12C shows the individual release profiles for the dye and FIG. 12D shows an optimised release profile from a combination of the delivery systems.

### Example 7

### In vivo Studies

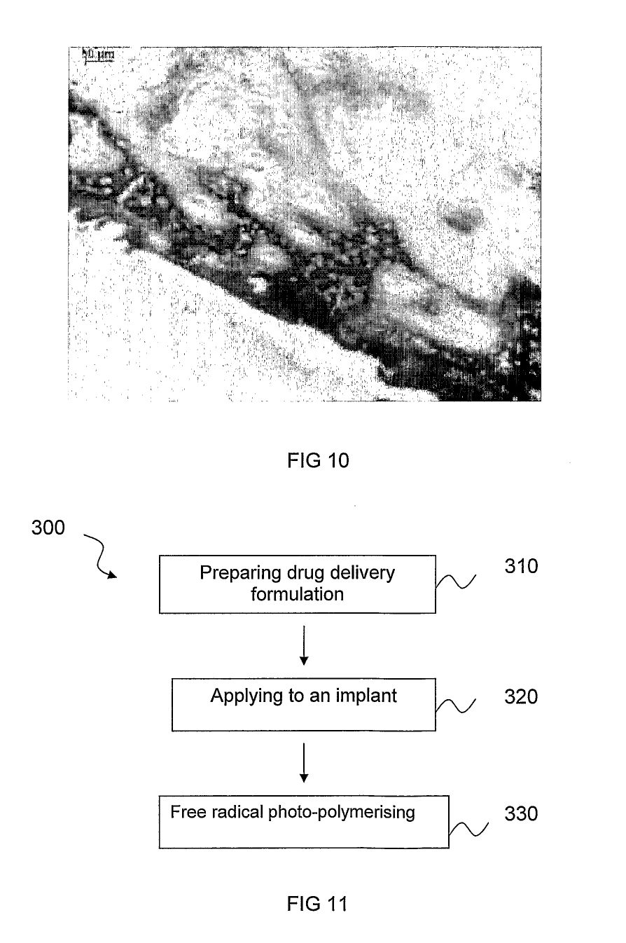

A rod was formed from 90MAA:7.5MMA:2.5TMPTM mole ratio network. The resulting material was implanted into a sheep femur. FIG. 9 shows a gross section of the femur, where the implant site is apparent. There is no evidence of inflammation or foreign body response. FIG. 10 is a histological section of the bone adjacent to the implant, showing no evidence of a foreign body response.

Hence, the compositions and implants of the present invention thus provide solutions to the aforementioned prior art problems by providing compositions and implants made from the compositions comprising sufficient mechanical strength for use in orthopaedic applications, that is biodegradable and therefore eliminated from the body once sufficient bone growth/fusion has occurred. Degradation is gradual and can be controlled according to the particular application to avoid premature degradation or degradation that is too slow. The compositions and implants made from the composition are biocompatible, reduce particulate debris and stress shielding and increase the fusion area. Furthermore, the compositions and implants can comprise one or more bioactive substances, such as growth factors to promote bone growth. Provision of the one or more biodegradable copolymer networks comprising one or more bioactive substances in cavities, recesses or voids in at least one surface of the implant and/or within the body of the implant provides a controlled, continuous and localized method of delivery of the bioactive substance(s), which is desirable to protect the bioactive substances, to achieve a steady rate of bone growth and to avoid doses of the bioactive substances that are either too high or too low that can negate the effect of the bioactive substances and/or lead to complications. Radiolucency of the implant also avoids artifacts in postoperative radiological assessment.

### Example 8

Pre-polymers in the form of methacrylated adipic anhydride (MAA) and methacrylated sebacic anhydride (MSA) and conversion of the acid to the anhydride was confirmed using ¹H nuclear magnetic resonance imaging (NMR) (Bruker, Alexandria, NSW) and FT-Infrared spectroscopy (Nicolet, Waltham Mass.). Formation of the pre-polymer was confirmed with the observation of NMR peaks at δ=5.8 and 6.2 ppm and FT-IR peaks at 1637 cm⁻¹.

The co-polymerisation behaviour was monitored by FT-Raman spectroscopy. Co-polymerisation was followed with consecutive FT-IR acquisitions with 100% conversion achieved between 10 and 30 hours depending on the ratio of MMA to MSA or MAA. Increasing the fraction of methacrylated anhydride slowed the rate of co-polymerisation.

The compressive mechanical properties of the biodegradable polymer composition were determined using an Instron 5567 (Bayswater, Victoria) and were measured as a function of the anhydride concentration, as shown in Table 1. The polymer composition was degraded in phosphate buffered solution (PBS) with various amounts of MAA and MSA.

The use of poly-L-lactic acid in lumbar interbody cages has been demonstrated to be mechanically feasible with the mechanical strength of the cage material reported to be 93 MPa (van Dijk, M. et al., The use of poly-L-lactic acid in lumbar interbody cages: design and biomechanical evaluation in vitro, Eur Spine J, Vol 12:34-40, 2003). The results demonstrate that most compositions of the biodegradable copolymer network of the present invention exhibit controlled mechanical properties comparable with, or in excess of, those required for implants.

Throughout the specification the aim has been to describe the invention without limiting the invention to any one embodiment or specific collection of features. Persons skilled in the relevant art may realize variations from the specific embodiments that will nonetheless fall within the scope of the invention.

### TABLES

TABLE 1
Biopolymer mechanical properties

| Composition | Compressive | Composition | Compressive |
|---|---|---|---|
| (MAA wt %) | Strength (MPa) | (MSA wt %) | Strength (MPa) |
| 10 | 90 ± 9 | 10 | 140 ± 18 |
| 30 | 118 ± 5 | 30 | 115 ± 8 |
| 40 | 140 ± 10 | 40 | 58 ± 2 |
| 45 | 62 ± 14 | 45 | 39 ± 1 |

1. A biodegradable copolymer network for an implant, said biodegradable copolymer network comprising two or more pre-polymers having a functional group polymerisable by a free radical mechanism wherein:
one of the pre-polymers is a degradable pre-polymer having a hydrolytically cleavable functional group;
one of the pre-polymers is a non-degradable enhancing pre-polymer; and
said two or more pre-polymers are co-polymerized to form a biodegradable copolymer network with enhanced mechanical properties in the form of a compressive strength of at least 47 MPa.

2. The biodegradable copolymer network as claimed in claim 1, wherein the degradable pre-polymer comprises one or more functional groups selected from the group consisting of acrylates, methacrylates and alkenes.

3. The biodegradable copolymer network as claimed in claim 1, wherein the non-degradable enhancing pre-polymer comprises functional groups selected from the group consisting of acrylates, methacrylates and alkenes.

4. The biodegradable copolymer network as claimed in claim 1, wherein the hydrolytically cleavable functional group of the degradable pre-polymer is selected from the functional groups consisting of anhydrides, esters, carbonates, amino carbonates and ortho-esters.

5. The biodegradable copolymer network as claimed in claim 1, wherein two or more pre-polymers are selected from the group consisting of a di-methacrylated acid anhydride and/or a tri-methacrylated acid anhydride.

6. The biodegradable copolymer network as claimed in claim 5, where the degradable pre-polymer comprises a di-methacrylated anhydride of a di carboxylic acid.

7. The biodegradable copolymer network as claimed in claim 5, where the degradable pre-polymer comprises a trimethacrylated anhydride of a tri carboxylic acid.

8. The biodegradable copolymer network as claimed in claim 5, where the degradable pre-polymer comprises a di-methacrylated anhydride of a di carboxylic acid and a tri methacrylated anhydride of a tri carboxylic acid.

9. The biodegradable copolymer network as claimed in claim 1, wherein the non-degradable enhancing pre-polymer is methyl methacrylate.

10. The biodegradable copolymer network as claimed in claim 1, wherein the biodegradable copolymer network further comprises at least one inorganic reinforcement substance.

11. The biodegradable copolymer network as claimed in claim 10, wherein the at least one inorganic reinforcement substance is selected from the group consisting of calcium hydroxyapatite and barium hydroxyapatite.

12. A medical implant or device prepared from the biodegradable copolymer network as claimed in claim 1.

13. A medical implant or device comprising two or more cavities at least partially filled with a first biodegradable copolymer network according to claim 1, wherein said first biodegradable copolymer network comprises one or more bioactive substances.

14. The medical implant or device as claimed in claim 13, wherein the two or more cavities further comprise a second biodegradable copolymer network selected from the group consisting of PLGA, PCL and polyanhydride.

15. The medical implant or device as claimed in claim 14, wherein the first biodegradable copolymer network and the second biodegradable copolymer network degrade at different rates.

16. The medical implant or device as claimed in claim 14, wherein the first biodegradable copolymer network and the second biodegradable copolymer network have different elution profiles for the bioactive substances such that the release profile can be controlled.

17. The medical implant or device as claimed in claim 13, wherein the one or more bioactive substances are selected from the group consisting of growth factors, peptides, antibiotics and pharmceuticals.

18. The medical implant or device as claimed in claim 13, wherein the one or more bioactive substances are polymerized in said two or more cavities.

19. The medical implant or device as claimed in claim 13, wherein the medical implant or device is at least partially formed from the first biodegradable copolymer network.

20. The medical implant or device as claimed in claim 19, wherein the remainder of the implant or device is formed from one or more non-degradable materials selected from the group consisting of calcium hydroxyapatite, barium hydroxyapatite, titanium and PEEK/carbon fibre composite.

21. The medical implant or device as claimed in claim 13, wherein the first biodegradable copolymer network further comprises a radio-opacifier selected from the group consisting of hydroxyapatite and barium hydroxyapatite.

22. A method of administering a bioactive substance in a controlled, continuous and localized manner, said method including the step of administering an implant, said implant comprising,
a biodegradable copolymer network comprising two or more pre-polymers having a functional group polymerisable by a free radical mechanism, wherein
one of the pre-polymers is a degradable pre-polymer having a hydrolytically cleavable functional group;
one of the pre-polymers is a non-degradable enhancing pre-polymer;
said two or more pre-polymers are co-polymerized to form a biodegradable copolymer network with enhanced mechanical properties in the form of a compressive strength of at least 47 MPa; and
one or more cavities are at least partially filled with the biodegradable copolymer network further comprising one or more bioactive substances.

23. A method of administering a bioactive substance in a controlled, continuous and localized manner, said method including the step of administering an implant to an animal, said implant comprising one or more cavities at least partially filled with a biodegradable copolymer network comprising one or more bioactive substances, said biodegradable copolymer network comprising two or more pre-polymers having a functional group polymerisable by a free radical mechanism, wherein
one of the pre-polymers is a degradable pre-polymer having a hydrolytically cleavable functional group;
one of the pre-polymers is a non-degradable enhancing pre-polymer; and said two or more pre-polymers are co-polymerized to form a biodegradable copolymer network with enhanced mechanical properties in the form of a compressive strength of at least 47 MPa; and
wherein said implant is formed from a predominantly non-biodegradable copolymer network.

24-25. (canceled)
